(19) United States
(12) Patent Application Publication
(10) Pub. No.: US 2022/0211932 A1
(43) Pub. Date: Jul. 7, 2022

(54) Tissue Interface With Integral Fluid-Control Layer

(71) Applicant: KCI Licensing, Inc., San Antonio, TX (US)

(72) Inventors: Timothy Mark ROBINSON, Shillingstone (GB); Christopher Brian LOCKE, Bournemouth (GB); Justin Alexander LONG, Lago Vista, TX (US)

(21) Appl. No.: 17/604,802

(22) PCT Filed: Mar. 16, 2020
(86) PCT No.: PCT/US2020/022928

§ 371 Date: Oct. 19, 2021

Related U.S. Application Data
(60) Provisional application No. 62/849,492, filed on May 17, 2019.

(51) Int. Cl. A61M 1/00; A61F 13/02; B29C 44/02; B29C 44/56; B29C 44/58

(52) U.S. Cl. CPC A61M 1/91; A61F 13/0216; A61F 13/0289; B29L 2031/753; B29C 44/5663; B29C 44/58; B29C 44/02

(57) ABSTRACT

Tissue dressing materials and associated methods of forming such dressing materials are disclosed. In one example embodiment, a dressing material may include a foam and a film formed on a first side of the foam. The film may be integrally-formed on the side of the foam. The film may include a plurality of apertures. A method of making the dressing material may include placing a pre-polymer mixture and water into a mold to create the dressing material comprising a foam and an integral film on at least one surface of the foam. The method may further include forming a plurality of openings in at least the integral film, with the openings being formed within the mold or subsequent to removal of the dressing material from the mold.

### RELATED APPLICATIONS

The present application claims priority to U.S. Provisional Patent Application No. 62/849,492, entitled “Tissue Interface with Integral Fluid-Control Layer,” filed May 17, 2019, which is incorporated herein by reference for all purposes.

### TECHNICAL FIELD

The invention set forth in the appended claims relates generally to tissue treatment systems and more particularly, but without limitation, to dressings and methods of making dressings for tissue treatment that may be applicable for use with negative-pressure therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for preparing dressings for treating tissue in a negative-pressure therapy environment are set forth in the appended claims. Illustrative embodiments are also provided to enable a person skilled in the art to make and use the claimed subject matter.

Exemplary tissue interfaces and dressing materials are disclosed, where the tissue interfaces and dressing materials may comprise a foam having an integral fluid-control layer. During traditional foam manufacturing processes film skins may be formed, however the film skins are typically skived off and discarded. In contrast, the tissue interfaces and/or dressing materials disclosed herein may retain the film skin as an integral layer on the foam material during manufacturing according to the disclosed methods. The film layer may be smooth and may be perforated, fenestrated, slotted, slit, laser cut, or otherwise made to have openings. Additionally, since the film skin remains on the foam material as an integral film layer of the tissue interface, the film layer may be perforated or fenestrated, with the perforations or fenestrations also at least partially extending through the foam material of the tissue interface in some embodiments.

For example, in some embodiments, a method for manufacturing a tissue interface or dressing material may comprise placing a pre-polymer mixture and water into a mold in order to generate a reaction to create a foam and an integral film on at least one surface of the foam. The method may further comprise forming a plurality of perforations in the integral film of the dressing material. The method may additionally comprise placing a secondary film material in the mold prior to placing the pre-polymer mixture and water into the mold. In some embodiments, the mold may be an open tray, and the method may further comprise skiving a top surface of the foam formed in the open tray.

In additional embodiments, a tissue interface may comprise a foam having a first side, a second side, and a fluid-control layer on the first side, and may further comprise a plurality of apertures extending through the fluid-control layer. In some instances, the foam may comprise a reticulated foam. The plurality of apertures may comprise linear fenestrations or perforations.

In further embodiments, a system for treating a tissue site may comprise a tissue interface and a cover. The tissue interface may comprise a polymer foam and a fluid-control layer. The cover may comprise a polymer drape adapted to be positioned over a second side of the polymer foam. The fluid-control layer may comprise a film integrally-formed on a first side of the polymer foam. The system may further include a dressing interface adapted to be coupled to the cover and a negative-pressure source adapted to be fluidly connected to the tissue interface through the dressing interface.

In yet further embodiments, a method for manufacturing a tissue interface may include creating a dressing material comprising a foam having a first side, a second side, and a fluid-control layer on the first side, and may further include forming a plurality of apertures in the fluid-control layer of the dressing material. The dressing material may comprise polyurethane. In some embodiments, forming the plurality of apertures may include using a blade to make fenestrations in the fluid-control layer. In some other embodiments, forming the plurality of apertures may include using one or more pins to make perforations in the fluid-control layer. In some embodiments, at least some of the plurality of apertures may extend through at least a portion of the foam.

In still further embodiments, a method of manufacturing a tissue interface or dressing material may include preparing a polymer mixture suitable for making a foam, extruding the polymer mixture to form a foam having a film formed on external surfaces of the foam, and forming a plurality of perforations in the film. In some examples, the foam may comprise a polyurethane foam or a polyethylene foam.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is an assembly view of an example of a tissue interface, illustrating details that may be associated with some example embodiments;

FIG. 2 is a schematic view, illustrating some additional details that may be associated with a portion of some example embodiments of the tissue interface of FIG. 1;

FIG. 3 is a flowchart of an exemplary method of forming the tissue interface of FIG. 1, illustrating details that may be associated with some example embodiments;

FIG. 4 is a schematic view of an example of a tissue interface positioned within a mold used during the formation of the tissue interface, illustrating details that may be associated with some example embodiments;

FIG. 5 is an assembly view of an example of a dressing that may incorporate the tissue interface of FIG. 1, according to some illustrative embodiments; and

FIG. 6 is a functional block diagram of an example embodiment of a therapy system that can provide negative-pressure treatment in accordance with this specification.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

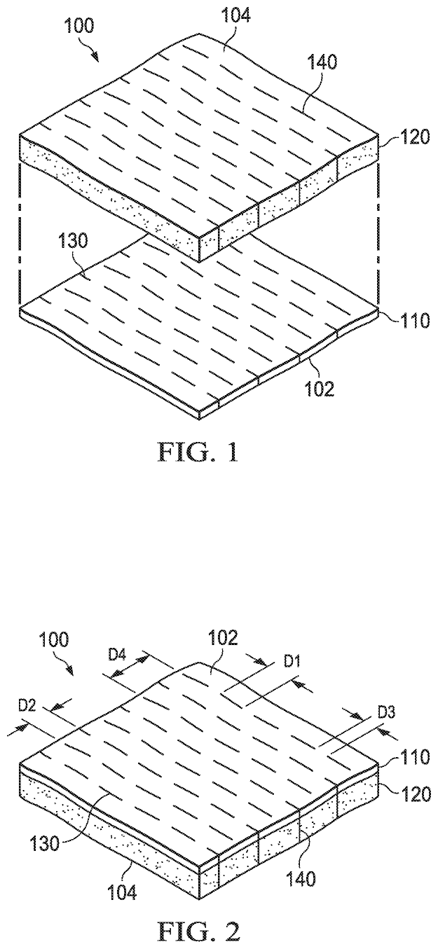

FIG. 1 is an assembly view of an example of a tissue interface 100 that can be applied to a tissue site. The tissue interface 100 can be generally adapted to partially or fully contact a tissue site. If the tissue site is a wound, for example, the tissue interface 100 may partially or completely fill the wound, or may be placed over the wound. The tissue interface 100 may have a first side 102 and a second side 104. The tissue interface 100 may be a single structure; however some examples of the tissue interface may comprise two different portions, or layers, that may be integral to the single structure. For example, the tissue interface 100 may include a first layer 110 and a second layer 120. In some embodiments, the first layer 110 may comprise a polymeric film, and the second layer 120 may comprise a polymeric foam. For example, the first layer 110 may comprise a polymeric film that is integrally formed on a surface of a polymeric foam of the second layer 120 during manufacture of the foam of the second layer 120. In some embodiments, the first layer 110 may be adapted to be placed against a tissue site, such as a wound and surrounding peri-wound area.

The tissue interface 100 may take many forms, and may have many sizes, shapes, or thicknesses, depending on a variety of factors, such as the type of treatment being implemented or the nature and size of a tissue site. While the tissue interface 100 is shown in FIG. 1 to have substantially a square shape, the tissue interface 100 and included layers may be any number of different shapes, based on the particular anatomical needs of a tissue site. For example, the tissue interface 100 and included layers may have a square, rectangular, oval, circular, hexagonal, or other shape. For example, the size and shape of the tissue interface 100 may be adapted to the contours of deep and irregularly-shaped tissue sites.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue.

The first layer 110 may comprise or consist essentially of a means for controlling or managing fluid flow, such as a fluid-control layer. In some embodiments, the first layer 110 may comprise or consist essentially of a liquid-impermeable material. For example, the first layer 110 may comprise or consist essentially of a non-porous polymer film. A first side of the first layer 110 that forms the first side 102 of the tissue interface 100 may have a smooth or matte surface texture in some embodiments. In some embodiments, variations in surface height on the first side 102 of the tissue interface 100 may be limited to acceptable tolerances, for example, with height variations limited to 0.2 millimeters over a centimeter.

In some embodiments, the first layer 110 may comprise or consist essentially of a polymeric film that is integral to the overall structure of the tissue interface 100. In some embodiments, the first layer 110 may comprise or consist essentially of a hydrophilic polymeric film, while in additional or alternative embodiments, the first layer 110 may comprise or consist essentially of a hydrophobic polymeric film. In some embodiments, the first layer 110 may comprise or consist essentially of a polyurethane film. For example, the first layer 110 may comprise a polyurethane film that is formed on a surface of a polyurethane foam during manufacture of the second layer 120, making the polyurethane film of the first layer 110 integral to the polyurethane foam of the second layer 120. Other suitable polymeric materials for forming the first layer 110 and the second layer 120 of the tissue interface may include silicones; elastomeric polyesters, for example HYTREL® elastomers; polyether copolymers, such as Pebax® elastomers; and isocyanate-free polyurethanes (amineoplast/carbamate copolymers; polycarbamate/polyamine materials; polycarbamate/polyaldehyde materials).

The first layer 110 may have material properties that make it conducive to being applied against a tissue site. For example, the first layer 110 may have a thickness of between 20 microns and 100 microns. In some embodiments, the hydrophobicity of the first layer 110 may be modified or enhanced with a hydrophobic coating of other materials, such as silicones and fluorocarbons, either as coated from a liquid or plasma coated.

In some embodiments, the first layer 110 may comprise an adhesive coating, which may be exposed on the first side 102 of the tissue interface 100. In some embodiments, the adhesive coating may comprise a low-tack medically-acceptable adhesive. For example, the adhesive coating may comprise a silicone gel, a polyurethane gel, or a low-tack acrylic adhesive. In some instances, the adhesive coating may have a low coat weight, such as between 25 grams per square meter and 100 grams per square meter, which may maintain a high moisture-vapor transmission rate (MVTR) of the first layer 110. The thickness of the adhesive coating may be tailored to balance the need to provide a good seal with the tissue site, while also maintaining a high MVTR. The adhesive coating may also be pattern coated on the first layer 110 in order to maintain the high MVTR of the first layer 110. The adhesive coating may assist with keeping the tissue interface 100 in place during application, which may be helpful to the user while finalizing the placement of the tissue interface 100 and sealing the tissue interface 100 to the tissue site.

As illustrated in the example of FIG. 1, the first layer 110 may have one or more openings 130, which may be distributed uniformly across the first layer 110 in some embodiments. The openings 130 may be bi-directional and pressure-responsive. For example, each of the openings 130 generally may comprise or consist essentially of an elastic passage that is normally unstrained to substantially reduce liquid flow, and can expand or open in response to a pressure gradient. The openings 130 may be in the form of fenestrations of perforations. In some embodiments, the openings 130 may comprise or consist essentially of fenestrations in the first layer 110. Fenestrations may be formed by removing material from the first layer 110, and may result in edges that are not deformed. In some alternative or additional embodiments, the openings 130 may comprise or consist essentially of perforations in the first layer 110. Perforations may be formed by removing material from the first layer 110. For example, perforations may be formed by cutting through the first layer 110. The amount of material removed and the resulting dimensions of the perforations may be an order of magnitude more than fenestrations, which may result in edges that are deformed. Additionally, in some embodiments, perforations may be formed by mechanical slitting then controlled uni- and/or bi-axial stretching of the film material of the first layer 110.

For example, some embodiments of the openings 130 may comprise or consist essentially of one or more slits, slots, or combinations of slits and slots in the first layer 110. In some examples, the openings 130 may comprise or consist of linear slots having a length less than 6 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, ultrasonics, or other heat means, for example. The linear slits or slots may be spaced apart by about 2 to 4 millimeters along their length and from side-to-side.

The second layer 120 generally comprises or consists essentially of a manifold or a manifold layer, which provides a means for collecting or distributing fluid across the tissue interface 100 under pressure. For example, the second layer 120 may be adapted to receive negative pressure from a source and distribute negative pressure through the tissue interface 100, which may have the effect of collecting fluid from across a tissue site and drawing the fluid toward the source. In some illustrative embodiments, the second layer 120 may comprise a plurality of pathways, which can be interconnected to improve distribution or collection of fluids. In some embodiments, the second layer 120 may comprise or consist essentially of a porous material having interconnected fluid pathways. For example, cellular foam, open-cell foam, reticulated foam, and other types of foam materials generally include pores, edges, and/or walls adapted to form interconnected fluid channels.

In some embodiments, the second layer 120 may comprise a hydrophilic or hydrophobic polymeric foam. For example, the second layer 120 may comprise or consist essentially of a polymeric foam, such as a polyurethane foam. For example, the second layer 120 may comprise or consist essentially of reticulated polyurethane foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of second layer 120 may also vary according to needs of a prescribed therapy. The 25% compression load deflection of the second layer 120 may be at least 0.35 pounds per square inch, and the 65% compression load deflection may be at least 0.43 pounds per square inch. In some embodiments, the tensile strength of the second layer 120 may be at least 10 pounds per square inch. The second layer 120 may have a tear strength of at least 2.5 pounds per inch. In some embodiments, the second layer 120 may be foam comprised of polyols such as polyester or polyether, isocyanate such as toluene diisocyanate, and polymerization modifiers such as amines and tin compounds. In some embodiments, water and/or a low-boiling-point liquid may be added to the precursor materials of the foam to assist with generating gasses for formation of the foam. In some examples, the second layer 120 may be reticulated polyurethane foam such as found in GRANUFOAM™ dressing or V.A.C. VERAFLO™ dressing, both available from Kinetic Concepts, Inc. of San Antonio, Tex.

In embodiments where the second layer 120 is hydrophilic, the second layer 120 may also wick fluid away from a tissue site, while being able to continue to distribute a negative pressure to the tissue site. The wicking properties of the second layer 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The thickness of the second layer 120 may also vary according to needs of a prescribed therapy. For example, the thickness of the second layer 120 may be decreased to reduce tension on peripheral tissue. The thickness of the second layer 120 can also affect the conformability of the second layer 120 and the tissue interface 100. In some embodiments, a thickness in a range of about 4 millimeters to 50 millimeters may be suitable, and in some more specific embodiments, the second layer 120 may have a thickness between 6 millimeters and 10 millimeters.

As illustrated in the example of FIG. 1, the second layer 120 may include a plurality of apertures 140, which may be distributed uniformly across the second layer 120. The apertures 140 may be in the form of fenestrations or tears through a portion or the entire thickness of the second layer 120. For example, the second layer 120 may comprise a reticulated polyurethane foam having apertures 140 in the form of finely-cut linear fenestrations. In some examples, the apertures 140 may comprise or consist of linear fenestrations having a length of between 1 millimeter and 6 millimeters, and a width less than 1 millimeter. In some embodiments, a length of about 3 millimeters may be particularly suitable for many applications, and a tolerance of about 0.1 millimeters may also be acceptable. The apertures 140 may be spaced apart by about 2 millimeters to 4 millimeters along their length and from side-to-side between the adjacent rows of the apertures 140, in some examples.

The apertures 140 of the second layer 120 may correspond to or be aligned with at least some of the openings 130 in the first layer 110. In some embodiments, the apertures 140 of the second layer 120 may be formed simultaneously with the formation of the openings 130 in the first layer 110. For example, the openings 130 in the first layer 110 and the apertures 140 in the second layer 120 may be formed during the formation of the first layer 110 and the second layer 120. For example, one or more techniques for forming the first layer 110 integrally with the second layer 120 may use molds that include projections such as pins to preserve open spaces or voids within both the first layer 110 and the second layer 120. The projections may result in the openings 130 and the apertures 140 in the first layer 110 and the second layer 120, respectively, when the tissue interface 100 is formed and removed from the mold. Other forms of cutting mechanisms may be used to form either or both of the openings 130 and apertures 140, for example using a knife or other blade, laser cutting, or ultrasonic cutting. For example, a knife or other blade may be used to simultaneously make fine cuts through both the first layer 110 and the second layer 120. In some embodiments, a cutting instrument may be used to make cuts completely through the first layer 110 to form the openings 130, but may only partially cut through the thickness of the second layer 120. In such embodiments, the apertures 140 may extend only partially through the thickness of the second layer 120. For example, unlike the illustrative example of FIG. 1, the apertures 140 may not extend all the way through the thickness of the second layer 120 to reach the second side 104 of the tissue interface 100.

In some embodiments, two or more layers of the tissue interface 100 may be coextensive. For example, the first layer 110 may be flush with the edges of the second layer 120. In some embodiments, the tissue interface 100 may be sized by simultaneously tearing or cutting through the integral first layer 110 and second layer 120.

FIG. 2 is a schematic view of a portion of the tissue interface 100 of FIG. 1 as assembled, showing further details that may be viewed from the first side 102 of the tissue interface 100, according to some illustrative embodiments. More specifically, the view of the first side 102 of the tissue interface 100 of FIG. 2 illustrates some additional details with respect to the openings 130 of the first layer 110 of the tissue interface 100. As illustrated in the example of FIG. 2, the openings 130 may each consist essentially of one or more linear slots having a length D₁, which may be about 3 millimeters. FIG. 2 additionally illustrates an example of a uniform distribution pattern of the openings 130. In FIG. 2, the openings 130 are substantially coextensive with the first layer 110 and are distributed across the first layer 110 in a grid of parallel rows and columns in which the slots are also mutually parallel to each other. In some embodiments, the rows may be spaced by a distance D₂, which may be about 3 millimeters on center, and the openings 130 within each of the rows may be spaced by a distance D₃, which may be about 3 millimeters on center as illustrated in the example of FIG. 2. The openings 130 in adjacent rows may be aligned or offset. For example adjacent rows may be offset, as illustrated in FIG. 2, so that the openings 130 are aligned in alternating rows and separated by a distance D₄, which may be about 6 millimeters. The spacing of the openings 130 may vary in some embodiments to increase the density of the openings 130 according to therapeutic requirements. Although not shown in FIG. 2, the openings 130 of the first layer 110 may be arranged in a variety of different patterns. For example, in some alternative embodiments, the openings 130 may be arranged in a grid with perpendicular rows. In some additional embodiments, the openings 130 may be arranged in both parallel and perpendicular rows. In some further embodiments, the openings 130 may be arranged in geometric patterns or shapes. As also shown in FIG. 2, the apertures 140 of the second layer 120 may be arranged in rows or other patterns corresponding to the arrangement of the openings 130 of the first layer 110. The openings 130 of the first layer 110 and the apertures 140 of the second layer 120 may be simultaneously formed. Although not shown in FIG. 2, in some alternative embodiments, the apertures 140 may only partially extend through the thickness of the second layer 120 and do not extend to the second side 104 of the tissue interface 100. Additionally, in some alternative embodiments, the second layer 120 may not have apertures 140.

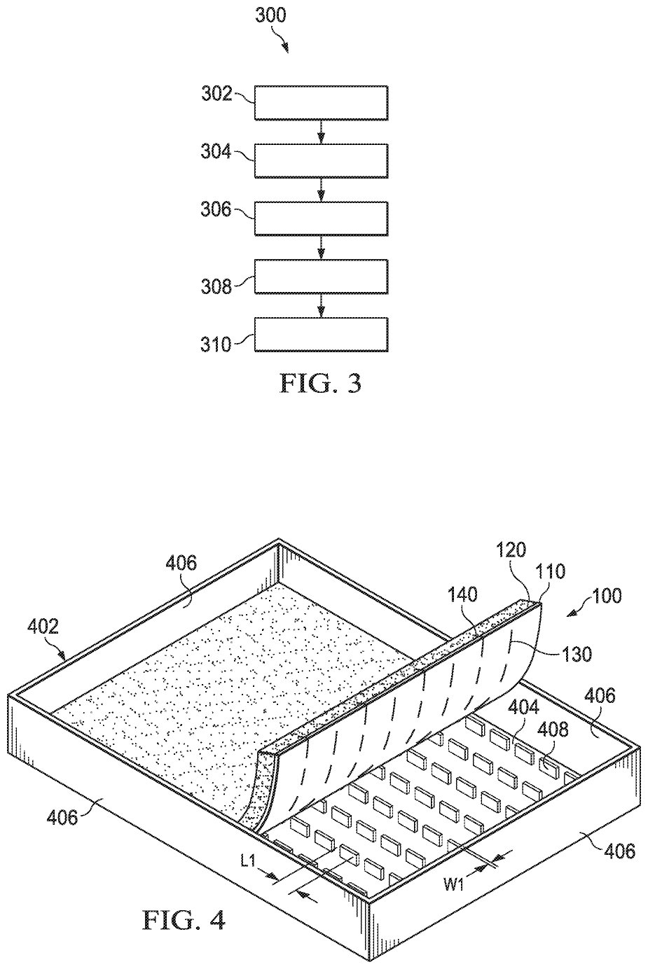

FIG. 3 is a flowchart of an example method 300 for forming some embodiments of the tissue interface. For example, forming the tissue interface 100 may begin at step 302 with preparing a pre-polymer mixture for use in creating a foam. In some embodiments, the pre-polymer mixture may comprise the ingredients for forming a polyurethane foam when mixed with water. For example, the pre-polymer mixture may comprise isocyanate and polyol. In some additional embodiments, the pre-polymer mixture may comprise the ingredients for forming a polyethylene foam when mixed with low-boiling-point liquids or pressurized gases, and accordingly the pre-polymer mixture may comprise molten polyethylene and low-boiling-point liquids such as fluorocarbons, pressurized gasses such as nitrogen, and/or chemical blowing agents such as citric acid and carbonate or bicarbonate salts. The method 300 may further include, at step 304, placing a pre-polymer mixture and additional ingredient, such as water in some embodiments, into a mold in order to generate a reaction to create the foam. In some embodiments, step 304 may comprise injecting the pre-polymer mix and water into a closed mold. As the ingredients of the mixture react within the mold, the foam may be generated. As the foam is formed, a film may also develop on one or more surfaces of the foam. For example, as the foam contacts one or more walls of the mold, the relative temperature of the portion, or surface, of the foam contacting the wall of the mold is reduced, thereby retarding or stopping the reaction of ingredients forming the foam. As a result, an integral film skin may be formed on the surfaces of the foam contacting the wall of the mold, with the integral film skin in effect being a portion of foam having a very high density, for example, a much higher density than the remainder of the generated foam. In some instances, the integral film skin may have a sufficiently high density such that it is essentially without pores. In some examples where the foam is a polyurethane foam, the integral film skin may, in effect, be in the form of a polyurethane elastomer. The integral film skin may provide the fluid-control layer of the tissue interface.

A variety of molds may be used as part of step 304 of the method 300. For example, a variety of shapes and sizes of molds may be used to manufacture different forms of the tissue interface 100 for use with various sizes and anatomical locations of a tissue site. For example, in addition to standard shapes, such as square or rectangular cuboids, molds may be used that correspond to anatomical shapes, such as feet, hands, breasts, sacral regions, etc. Additionally, since the molding of the foams may be performed using relatively low-pressure processes, relatively inexpensive, disposable molds may be used in some embodiments. For example, some disposable molds may be formed from castable polymers or ceramics, such as plaster of paris, which may allow for the fabrication of customizable molds based on an individual patient anatomy corresponding to a tissue site.

The interior surfaces of the molds used in the method 300 for the formation of the tissue interface 100 may also be tailored based on specific applications of the tissue interface 100. For example, while in some embodiments the interior surfaces of the molds may be smooth, additional or alternative embodiments may include molds having features for forming embossed textures on one or more surfaces of the tissue interface 100. For example, embossed features may be formed on the first layer 110 forming the first side 102 of the tissue interface 100. Embossed or textured features may also be included on one or more interior surfaces of the mold so as to impart such features on other portions of the tissue interface, such as an upper side of the foam of the second layer 120 of the tissue interface. Embossed features may be particularly useful for sizing the tissue interface 100, as well as for placing and orienting the tissue interface 100 on a tissue site. Additionally, embossed features may assist with fluid removal functionality of the tissue interface 100 when applied to a tissue site.

The method 300 may additionally include, at step 306, forming the openings 130 in the integral film, or fluid-control layer, of the first layer 110. Depending on the particular embodiment, the apertures 140 may also be formed in the foam of the second layer 120. The openings 130 and, if desired, the apertures 140 may be formed either during the formation of the first layer 110 and second layer 120 within the mold, or as a process following the formation of the layers of the tissue interface 100. In some embodiments, perforations or fenestrations may be made in the first layer 110 and second layer 120 to form the openings 130 and the apertures 140, respectively, by one or more pins, rods, or blades positioned within or formed as part of the mold. For example, as the ingredients react to form the foam of the second layer 120 and integral film of the first layer 110, spaces with both the foam and integral film layer may be preserved by the pins, rods, or blades. Additionally or alternatively, the formed foam of the second layer 120 with the integral film of the first layer 110 may be removed from the mold, and perforations or fenestrations may be formed in either or both of the film of the first layer 110 and foam of the second layer 120 using one or more blades, pins, rods, or other appropriate cutting instrument and/or mechanism.

The tissue interface 100 may be formed or manufactured in a range of sizes for use in a variety of dressings having different sizes. Additionally, the tissue interface 100, once formed, may be cut or sized, at step 308, to approximately the size of a tissue site. In some embodiments, it may be appropriate or beneficial to size the tissue interface 100 so that it covers a perimeter area of a tissue site. Additionally, the tissue interface 100 may be provided in a range of thicknesses. For example, some embodiments of the tissue interface 100 may have a thickness ranging from approximately 3 mm to 50 mm.

The method 300 may further include, at step 310, reticulating the foam portion of the tissue interface 100. In some embodiments, once the foam of the second layer 120 and the integral film of the first layer 110 have been formed in the mold, and the openings 130 have been formed in the integral film, the reticulation process may be performed. In some embodiments of the method 300, the foam may be reticulated before being removed from the mold. Additionally or alternatively, the tissue interface 100 may be removed from the mold and stacked within a reticulation chamber where the reticulation process may be conducted. Regardless of where the tissue interface 100 is positioned during the reticulation process, the openings 130 formed in the integral film of the first layer 110 as part of step 306 may allow the reticulation gas to enter the pore structure of the foam of the second layer 120 of the tissue interface 100 and also permit the escape of the combustion gases, such as water vapor, from the foam during the reticulation process. As an alternative to using a gas reticulation procedure for creating the porosity in the foam of the second layer, one or more cell openers may be used to introduce weakness into the cell walls of the polymer foam during its formation. Example cell openers may include calcium carbonate, nanoparticles, and/or anti-foaming agents such as siloxanes and polyethylene oxides.

In some additional embodiments, since both the integral film layer, or fluid-control layer, and the foam portion of the tissue interface 100 may be perforated or fenestrated, the perforations in the foam may act as manifolding passageways. Therefore, in some instances, the step of reticulating the foam portion may not be necessary, as the channels formed by the perforations or fenestrations in the foam may enable a non-reticulated foam to have sufficient capability for fluid handling and communication of air and fluids in negative-pressure therapy applications.

In addition to using closed molds, various other molding techniques may be used to form one or more embodiments of the tissue interface 100. For example, in some additional embodiments, foam-slab casting methods involving a foam mixture being poured into a tray may be used to create a foam for the second layer 120 in the form of a relatively thin foam sheet with an integral film or skin for the first layer 110. For example, once poured into the mold, the pre-polymer mixture and water may generate a relatively thin foam sheet with an integral film formed on the surface of the foam layer that forms in contact with the bottom of the tray mold. Additionally, during formation of the foam layer, the upper surface, such as the surface in contact with the air or atmosphere, may be skived to remove any intermittent- or variable-density foam. Forming the foam of the second layer 120 with the integral film for the first layer 110 on one side of the second layer 120 may present a cost-effective method of making the tissue interface 100. It may be expected that the integral film of the first layer 110 will be placed in contact with a tissue site.

In some embodiments of the method 300, additional steps may include forming one or more surface features on the integral film of the first layer 110 of the tissue interface 100. For example, some embodiments may include using in-mold decoration techniques for providing additional or different layers or types of film, colored or patterned features, or surface-textured features to an outer surface of the integral film of the first layer 110 of the tissue interface 100. For example, such steps for accomplishing these one or more surface features may include placing a textured secondary or additional film into a mold prior to the injection of the foam ingredients into the mold. In additional or alternative embodiments, a secondary or additional material may first be spray-coated into the mold and allowed to form and cure into a film. For example, the material may be cured into a film such as by using ultraviolet light to cross-link the precursors of the film. The foam ingredients may then be injected into the mold to form the first layer 110 and second layer 120 of the tissue interface 100.

FIG. 4 is a schematic view of an example of a tissue interface 100 positioned within a mold during the formation of the tissue interface 100, according to some illustrative embodiments. In some embodiments, as shown in FIG. 4, a mold 402 may be an open mold, such as a tray mold. The mold 402 may have a variety of shapes and sizes, for example a square cuboid with a side removed, rectangular cuboid with a side removed, a cylinder with a side removed, or any other suitable shape for forming a tissue interface 100. For example, as shown in FIG. 4, the mold 402 may be a rectangular cuboid with a top side removed and having a bottom 404 and a plurality of sides 406. As also depicted in FIG. 4, the mold 402 may include a plurality of projections 408 extending upward from the bottom 404 of the mold 402.

The plurality of projections 408 may be used to form the perforations or fenestrations of the openings 130 in the first layer 110, during formation of the tissue interface 100. In some embodiments, if apertures 140 in the second layer 120 are desired, the plurality of projections 408 may also form the apertures 140. The plurality of projections 408 may be in the form of rods, pins, blades, or other form of projections. In some embodiments, each of the plurality of projections 408 may be a cylindrically-shaped projection, while in additional or alternative embodiments, each of the projections may have another shape such as a rectangular cuboid.

The projections 408 may have a variety of diameters or cross-sectional areas depending on the particular size of the perforations or fenestrations desired in the first layer 110 and second layer 120 of the tissue interface 100. For example, for cylindrically-shaped projections 408, each of the plurality of projections 408 may have a diameter of between about 2 mm and 6 mm. In other embodiments, as illustrated in FIG. 4, the projections 408 may be in the form of square or rectangular cuboids and may have a length Li, which may be between about 1 mm and 8 mm, and a width Wi, which may be between about 0.2 mm and 2 mm. In some additional embodiments, the shape of each of the plurality of projections 408 may be angled, such that the bottom portions of the projections 408 have a greater diameter or cross-sectional area than the top portions of the projections 408.

The plurality of projections 408 may occupy space within the volume of the mold 402, such that the liquid pre-cursor materials of the tissue interface 100 do not occupy the space reserved by the plurality of projections 408 during formation of the tissue interface 100. When the tissue interface 100 is formed and removed from the mold 402, perforations or fenestrations forming the openings 130 may exist in the first layer 110 of the tissue interface 100. Depending on the height of the plurality of projections 408 from the bottom 404 of the mold 402, the projections 408 may also extend far enough upwards so as to also form the perforations or fenestrations of the apertures 140 through at least a portion of the thickness of the second layer 120 of the tissue interface 100. For example, each of the plurality of projections 408 may have a height in a range of 1 mm to 10 mm. In some particular embodiments, the projections 408 may each have a height of between about 3 mm and 6 mm, which may be suitable for forming the openings 130 in the first layer 110 of the tissue interface 100, but not extend significantly above the first layer 110 to form perforations or fenestrations in the second layer 120. In some additional embodiments, the projections 408 may each have a height in a range of 4 mm to 8 mm, which may be suitable for forming both the openings 130 in the first layer 110 and the apertures 140 in the second layer 120 of the tissue interface 100. Furthermore, the plurality of projections 408 may include projections having different heights, for example a first group of projections where each of the projections of the first group has a greater height than each of the projections of a second group of projections. In such embodiments, the projections of the first group of projections having the greater height may form both openings 130 in the first layer 110 and apertures 140 in the second layer 120 of the tissue interface 100, while the projections of the second group of projections having the lesser height may form only openings 130 in the first layer 110.

The plurality of projections 408 may be distributed uniformly across the bottom 404 of the mold 402, or alternatively, may also be distributed in an arrangement for forming perforations or fenestrations in the one or more layers of the tissue interface 100 according to a pattern that may be suitable or ideal for particular applications of the tissue interface 100. For example, the plurality of perforations 408 may be arranged so that either a greater or lesser number of individual perforations 408 are located in either the center or peripheral portions of the mold 402. Overall, many combinations of sizes, heights, and arrangements of the plurality of projections 408 may be implemented to achieve the ideal arrangement of openings 130 in the first layer 110 as well as optionally apertures 140 in the second layer 120, depending on the particular design and intended application of the tissue interface 100.

Some embodiments of the tissue interface 100 may also be formed using other methods and manufacturing processes. For example, in some embodiments, the tissue interface 100 may be formed using an extrusion process. For example, the foam material forming the second layer 120 may be extruded with an outer integral skin forming the first layer 110 included on all sides of the extruded foam material. Once the extrusion process is completed, perforations or fenestrations may be formed in the extruded foam and film to form the openings 130 in the first layer 110 and the apertures 140 in the second layer 120 of the extruded materials of the tissue interface 100.

Additionally, in some further examples of the tissue interface 100, different combinations of materials for the film of the first layer 110 and the foam of the second layer 120 may be used. For example, some embodiments may include a first layer 110 comprising a polyethylene film, while the second layer 120 may comprise a polyurethane foam. In some instances, it may be advantageous to apply the polyethylene film of the first layer 110 in a mold, and then surface treat, perhaps with a bonding agent, the polyethylene film so that the polyurethane foam of the second layer 120 will bond to the polyethylene film. In some further embodiments, the first layer 110 may comprise both an outer polyethylene film and an inner polyurethane film with a bonding agent as a compatibility agent between the polyethylene and polyurethane films. Such a multi-component first layer 110 may be applied to the mold, and the mixture for forming the polyurethane foam may then be injected into the mold to form the second layer 120.

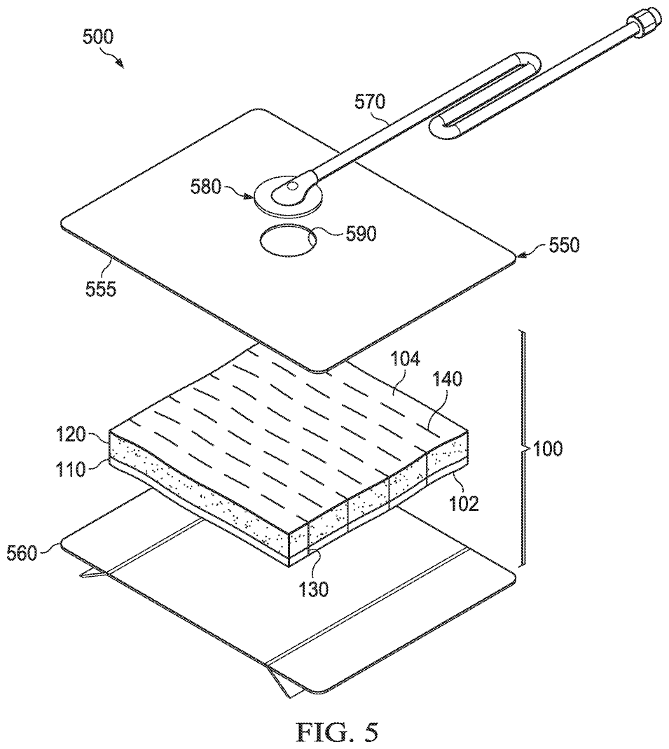

FIG. 5 is an assembly view of an example of a dressing 500 with the tissue interface 100 of FIG. 1. For example, the dressing 500 may include the tissue interface 100, along with additional components that may enable or particularly facilitate use of the dressing 500 and associated tissue interface 100 with negative-pressure therapy. As depicted in FIG. 5, the first layer 110 of the tissue interface 100 may form the first side 102 of the tissue interface 100, and the second layer 120 may form the second side 104 of the tissue interface 100. While the dressing 500, including the tissue interface 100, is shown in FIG. 5 to have a substantially square shape, the dressing 500 and included layers may be any number of different shapes, based on the particular anatomical needs of a tissue site. For example, the dressing 500 and included layers may have a square, rectangular, oval, circular, hexagonal, or other shape. Additionally, the dressing 500 may further include three-dimensional forms that may be shaped to address needs of specific types of tissue sites, such as breasts, hands, feet, sacral regions of a patient, or other wounds.

The dressing 500 may also include a cover 550, which may provide a bacterial barrier and protection from physical trauma. The cover 550 may also be constructed from a material that can reduce evaporative losses and provide a fluid seal between two components or two environments, such as between a therapeutic environment and a local external environment. The cover 550 may comprise or consist of, for example, an elastomeric film or membrane that can provide a seal adequate to maintain a negative pressure at a tissue site for a given negative-pressure source. The cover 550 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 550 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 550 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 550 may comprise INSPIRE 2301 having an MVTR (upright cup technique) of 2600 g/m²/24 hours and a thickness of about 30 microns.

In the example of FIG. 5, the dressing may further include an attachment device for attaching the cover 550 to an attachment surface, such as undamaged epidermis, a gasket, or another cover. The attachment device may take many forms. For example, an attachment device may comprise an adhesive 555, which may be a medically-acceptable, pressure-sensitive adhesive configured to bond the cover 550 to epidermis around a tissue site. In some embodiments, for example, some or all of the cover 550 may be coated with the adhesive 555, such as an acrylic adhesive, which may have a coating weight of about 25-65 grams per square meter (g.s.m.). Thicker adhesives, or combinations of adhesives, may be applied in some embodiments to improve the seal and reduce leaks. In some embodiments, such a layer of the adhesive 555 may be continuous or discontinuous. Discontinuities in the adhesive 555 may be provided by apertures or holes (not shown) in the adhesive 555. The apertures or holes in the adhesive 555 may be formed after application of the adhesive 555 or by coating the adhesive 555 in patterns on a carrier layer, such as the cover 550. Apertures or holes in the adhesive 555 may also be sized to enhance the MVTR of the dressing 500 in some example embodiments. Other example embodiments of an attachment device may include a double-sided tape, paste, hydrocolloid, hydrogel, silicone gel, or organogel.

As illustrated in the example of FIG. 5, in some embodiments, the dressing 500 may include a release liner 560 to protect the first side 102 of the tissue interface 100 and to protect the adhesive 555 prior to use of the dressing 500. The release liner 560 may also provide stiffness to assist with, for example, deployment of the dressing 500. The release liner 560 may be, for example, a casting paper, a film, or polyethylene. Further, in some embodiments, the release liner 560 may be a polyester material such as polyethylene terephthalate (PET) or similar polar semi-crystalline polymer. The use of a polar semi-crystalline polymer for the release liner 560 may substantially preclude wrinkling or other deformation of the dressing 500. For example, the polar semi-crystalline polymer may be highly orientated and resistant to softening, swelling, or other deformation that may occur when brought into contact with components of the dressing 500 or when subjected to temperature or environmental variations, or sterilization. Further, a release agent may be disposed on a side of the release liner 560 that is configured to contact the tissue interface 100. For example, the release agent may be a silicone coating and may have a release factor suitable to facilitate removal of the release liner 560 by hand and without damaging or deforming the dressing 500. In some embodiments, the release agent may be a fluorocarbon or a fluorosilicone, for example. In other embodiments, the release liner 560 may be uncoated or otherwise used without a release agent.

FIG. 5 also illustrates one example of a fluid conductor 570 and a dressing interface 580. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. As shown in the example of FIG. 5, the fluid conductor 570 may be a flexible tube, which can be fluidly coupled on one end to the dressing interface 580. The dressing interface 580 may be an elbow connector, as shown in the example of FIG. 5, which can be placed over an aperture 590 in the cover 550 to provide a fluid path between the fluid conductor 570 and the tissue interface 100. In some embodiments, the fluid conductor 570 may also include a fluid delivery conduit for use with instillation therapy. Further, in some embodiments, the dressing interface 580 may include multiple fluid conduits, such as a conduit for communicating negative pressure and a fluid delivery conduit. For example, the dressing interface 580 may be a V.A.C. VERAT.R.A.C.™ Pad or a SENSAT.R.A.C.™ Pad, available from KCI of San Antonio, Tex.

Individual components of the dressing 500, may be bonded or otherwise secured to one another with a solvent or non-solvent adhesive, or with thermal welding, for example, without adversely affecting fluid management. In some embodiments, one or more components of the dressing 500 may additionally be treated with an antimicrobial agent. For example, the tissue interface 100, the fluid conductor 570, the dressing interface 580, or other portion of the dressing 500 may additionally or alternatively be treated with one or more antimicrobial agents. Suitable agents may include, for example, metallic silver, PHMB, iodine or its complexes and mixes such as povidone iodine, copper metal compounds, chlorhexidine, or some combination of these materials. Additionally or alternatively, a portion of the tissue interface 100 may be coated with a mixture that may include citric acid and collagen, which can reduce bio-films and infections.

In use, the tissue interface 100 may be sized to a specific region or anatomical area corresponding to a tissue site through cutting or tearing, if not already customized during manufacture to the size of the target tissue site. The release liner 560 (if included) may be removed from the first side 102 of the tissue interface 100. The tissue interface 100 may then be sized if necessary. The tissue interface 100 may be cut or torn to an appropriate size without the individual layers that are integral to the tissue interface 100, such as the first layer 110 and the second layer 120, becoming separated from each other or falling apart.

Once the tissue interface 100 is sized and/or shaped to the area of the tissue site, the tissue interface 100 may be placed within, over, on, or otherwise proximate to the tissue site, particularly a surface tissue site and adjacent epidermis. The first layer 110 may be interposed between the second layer 120 and the tissue site. For example, the tissue interface 100 may be placed so that the first side 102 formed by the first layer 110 of the tissue interface 100 is positioned over a surface wound (including edges of the wound) and undamaged epidermis to prevent direct contact between the second layer 120 of the tissue interface 100 and the epidermis. Treatment of a surface wound or placement of the tissue interface 100 on a surface wound includes placing the tissue interface 100 immediately adjacent to the surface of the body or extending over at least a portion of the surface of the body. The cover 550 may then be placed over the second side 104 of the tissue interface 100 and sealed, using the adhesive 555, to an attachment surface surrounding the tissue site, such as adjacent epidermis, to enable a pneumatic seal around the tissue site. The dressing interface 580 may then be disposed over the aperture 590 of the cover 550. The fluid conductor 570 may be fluidly coupled to the dressing interface 580.

In some applications, a filler may also be disposed between a tissue site and the first layer 110 of the tissue interface 100. For example, if the tissue site is a surface wound, a wound filler may be applied interior to the peri-wound, and the tissue interface 100, specifically the first layer 110 forming the first side 102 of the tissue interface, may be disposed over the peri-wound and the wound filler. In some embodiments, the filler may be a manifold, such as an open-cell foam. The filler may comprise or consist essentially of the same material as the second layer 120 in some embodiments.

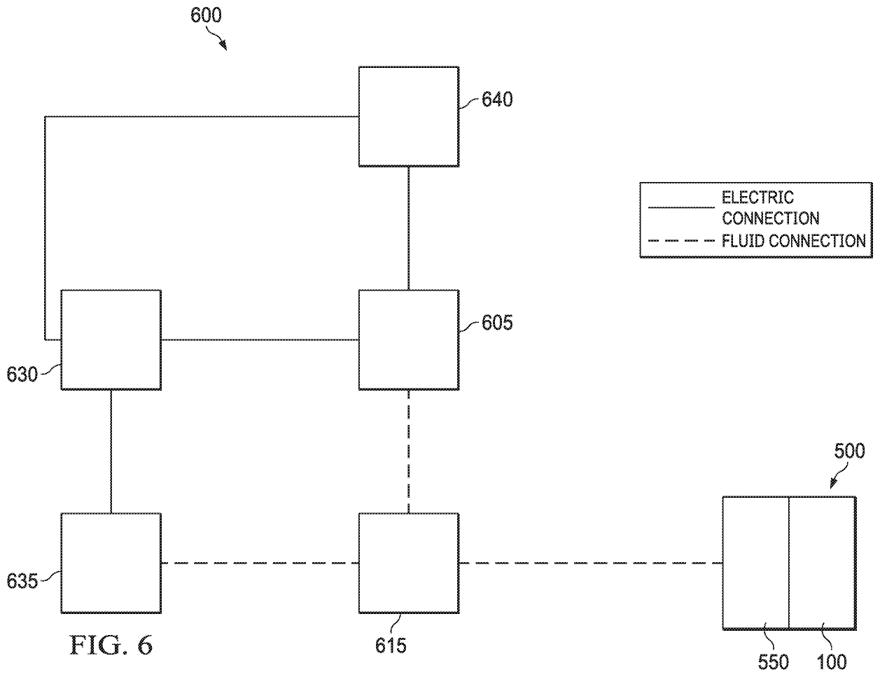

FIG. 6 is a simplified functional block diagram of an example embodiment of a therapy system 600 that can provide negative-pressure therapy to a tissue site with various embodiments of the tissue interface 100. The therapy system 600 may include a source or supply of negative pressure, such as a negative-pressure source 605, and one or more distribution components. A distribution component is preferably detachable and may be disposable, reusable, or recyclable. A dressing, such as the dressing 500, and a fluid container, such as a container 615, are examples of distribution components that may be associated with some examples of the therapy system 600. The container 615 is representative of a container, canister, pouch, or other storage compartment, which can be used to manage exudates and other fluids withdrawn from a tissue site. A fluid conductor is another illustrative example of a distribution component. Distribution components may also include or comprise interfaces or fluid ports to facilitate coupling and de-coupling other components. As illustrated in the example of FIG. 6, the dressing 500 may comprise or consist essentially of the tissue interface 100 and the cover 550.

The therapy system 600 may also include a regulator or controller, such as a controller 630. Additionally, the therapy system 600 may include sensors to measure operating parameters and provide feedback signals to the controller 630 indicative of the operating parameters. As illustrated in FIG. 6, for example, the therapy system 600 may include a first sensor 635 and a second sensor 640 coupled to the controller 630.

Some components of the therapy system 600 may be housed within or used in conjunction with other components, such as sensors, processing units, alarm indicators, memory, databases, software, display devices, or user interfaces that further facilitate therapy. For example, in some embodiments, the negative-pressure source 605 may be combined with the controller 630 and other components into a therapy unit.

In general, components of the therapy system 600 may be coupled directly or indirectly. Coupling may include fluid, mechanical, thermal, electrical, or chemical coupling (such as a chemical bond), or some combination of coupling in some contexts.

A negative-pressure supply, such as the negative-pressure source 605, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 605 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

A controller, such as the controller 630, may be a microprocessor or computer programmed to operate one or more components of the therapy system 600, such as the negative-pressure source 605. The controller 630 may control one or more operating parameters of the therapy system 600, which may include the power applied to the negative-pressure source 605, the pressure generated by the negative-pressure source 605, or the pressure distributed to the tissue interface 100, for example. The controller 630 is also preferably configured to receive one or more input signals, such as a feedback signal, and programmed to modify one or more operating parameters based on the input signals.

Sensors, such as the first sensor 635 and the second sensor 640, are generally known in the art as any apparatus operable to detect or measure a physical phenomenon or property, and generally provide a signal indicative of the phenomenon or property that is detected or measured. For example, the first sensor 635 and the second sensor 640 may be configured to measure one or more operating parameters of the therapy system 600. In some embodiments, the first sensor 635 may be a transducer configured to measure pressure in a pneumatic pathway and convert the measurement to a signal indicative of the pressure measured. The second sensor 640 may optionally measure operating parameters of the negative-pressure source 605, such as a voltage or current, in some embodiments.

In operation, the tissue interface 100 may be placed within, over, on, or otherwise proximate to a tissue site. The cover 550 may optionally be placed over the tissue interface 100 and sealed to an attachment surface near a tissue site. For example, the cover 550 may be sealed to undamaged epidermis peripheral to a tissue site. Thus, the dressing 500 can provide a sealed therapeutic environment proximate to a tissue site, substantially isolated from the external environment, and the negative-pressure source 605 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 100 in the sealed therapeutic environment can induce macro-strain and micro-strain in the tissue site. Negative pressure can also remove exudate and other fluid from a tissue site, which can be collected in container 615. For example, negative pressure applied through the tissue interface 100 can create a negative-pressure differential across the openings 130 in the first layer 110, which can open or expand the openings 130 from their resting state. For example, in some embodiments in which the openings 130 may comprise substantially closed fenestrations through the first layer 110, a pressure gradient across the fenestrations can strain the adjacent material of the first layer 110 and increase the dimensions of the fenestrations to allow liquid movement through them, similar to the operation of a duckbill valve. Opening the openings 130 can allow exudate and other liquid movement through the openings 130, through the second layer 120, and into the container 615. Changes in pressure can also cause the second layer 120 to expand and contract, and the first layer 110 may protect the epidermis from irritation caused by the movement of the second layer 120. The first layer 110 can also substantially reduce or prevent exposure of tissue to the second layer 120, which can inhibit growth of tissue into the second layer 120.

In some embodiments, the controller 630 may receive and process data from one or more sensors, such as the first sensor 635. The controller 630 may also control the operation of one or more components of the therapy system 600 to manage the pressure delivered to the tissue interface 100. In some embodiments, controller 630 may include an input for receiving a desired target pressure and may be programmed for processing data relating to the setting and inputting of the target pressure to be applied to the tissue interface 100. In some example embodiments, the target pressure may be a fixed pressure value set by an operator as the target negative pressure desired for therapy at a tissue site and then provided as input to the controller 630. The target pressure may vary from tissue site to tissue site based on the type of tissue forming a tissue site, the type of injury or wound (if any), the medical condition of the patient, and the preference of the attending physician. After selecting a desired target pressure, the controller 630 can operate the negative-pressure source 605 in one or more control modes based on the target pressure and may receive feedback from one or more sensors to maintain the target pressure at the tissue interface 100.

If the negative-pressure source 605 is removed or turned-off, the pressure differential across the openings 130 of the first layer 110 of the tissue interface 100 can dissipate, allowing the openings 130 to move to their resting state and prevent or reduce the rate at which exudate or other liquid can return to the tissue site through the first layer 110.

The systems, apparatuses, and methods described herein may provide significant advantages. Among other things, the complexity and cost of manufacturing the tissue interface 100 may be significantly reduced. For example, the tissue interface 100 may be a ready-to-use foam dressing material with an integral film layer that does not require additional processing, or lamination, to apply the film layer to the foam material. Additionally or alternatively, risk of de-lamination, particularly under potentially aggressive conditions at the tissue site, such as flexing, stretching, or even in conjunction with fluid instillation therapy, may be minimized or eliminated. Methods for manufacturing some embodiments of the tissue interface 100 may also be scaled-up for higher output and improved economics. Additionally, some embodiments of the tissue interface 100 may be manufactured to a specific anatomy without extrusion and cutting processes.

The tissue interface 100 having integral foam and film layers may also offer significant benefits during use or application of the tissue interface 100 to a tissue site. For example, the tissue interface 100 may have a smooth contact surface, which can significantly reduce or eliminate irritation to perimeter areas of intact tissue, such as epidermis adjacent to a tissue site.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the tissue interface 100 may be separated from other components for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A method for manufacturing a dressing material, comprising:
placing a pre-polymer mixture and water into a mold to generate a reaction that produces a foam and an integral film on at least one surface of the foam; and
forming a plurality of openings in the integral film of the dressing material.

2. The method of claim 1, further comprising placing a secondary film in the mold prior to placing the pre-polymer mixture and water into the mold.

3. The method of claim 2, wherein the secondary film comprises one or more of a patterned material, a colored material, and a plurality of textured features.

4. (canceled)

5. (canceled)

6. The method of claim 1, wherein the mold is an open tray and the method further comprises skiving a top surface of the foam formed in the open tray.

7. The method of claim 6, wherein the mold comprises a bottom side having a first surface facing an interior of the mold, and wherein the first surface is smooth.

8. The method of claim 1, wherein the pre-polymer mixture comprises isocyanate and polyol.

9. The method of claim 1, wherein the pre-polymer mixture comprises molten polyethylene and a low-boiling-point liquid.

10. (canceled)

11. (canceled)

12. The method of claim 1, wherein forming the plurality of openings comprises using a blade to make fenestrations in the integral film.

13. The method of claim 1, wherein forming the plurality of openings comprises using one or more pins to make the openings in the integral film.

14. The method of claim 1, wherein the plurality of openings extend through at least a portion of the foam.

15. The method of claim 1, wherein the plurality of openings are formed by a plurality of pins within the mold.

16. The method of claim 1, wherein the integral film has a thickness of between 20 μm and 100 μm, and wherein the foam has a thickness of between 4 mm and 50 mm.

17. (canceled)

18. The method of claim 1, wherein an interior surface of the mold comprises a plurality of embossed features.

19. The method of claim 1, wherein the mold comprises an anatomical shape corresponding to at least one of a breast, a hand, a sacral region, or a foot.

20. (canceled)

21. (canceled)

22. (canceled)

23. (canceled)

24. (canceled)

25. (canceled)

26. The method of claim 1, further comprising placing the dressing material in a reticulation chamber and applying a gas to form a plurality of pores in the foam.

27. A tissue interface, comprising:
a foam having a first side, a second side, and a fluid-control layer on the first side; and
a plurality of apertures extending through the fluid-control layer.

28. (canceled)

29. (canceled)

30. The tissue interface of claim 27, wherein the second side of the foam comprises a plurality of embossed features.

31. (canceled)

32. The tissue interface of claim 27, wherein the foam comprises a plurality of perforations.

33. (canceled)

34. (canceled)

35. The tissue interface of claim 27, wherein the tissue interface comprises an anatomical shape corresponding to at least one of a foot, a hand, a breast, or a sacral region.

36. (canceled)

37. (canceled)

38. (canceled)

39. (canceled)

40. (canceled)

41. (canceled)

42. (canceled)

43. The tissue interface of claim 27, wherein the plurality of apertures comprise linear fenestrations that are distributed across the fluid-control layer.

44. (canceled)

45. The tissue interface of claim 27, wherein the fluid-control layer comprises an adhesive surface, and wherein the adhesive surface comprises a silicone gel.

46. (canceled)

47. The tissue interface of claim 27, wherein the fluid-control layer comprises a liquid-impermeable film.

48. (canceled)

49. (canceled)

50. A system for treating a tissue site, comprising:
the tissue interface according to claim 27; and
a cover comprising a polymer drape adapted to be positioned over a second side of the foam.

51. The system of claim 50, wherein the fluid-control layer comprises a film integrally-formed on a first side of the foam.

52. (canceled)

53. (canceled)

54. (canceled)

55. The system of claim 50, further comprising:
a dressing interface adapted to be coupled to the cover; and
a negative-pressure source adapted to be fluidly connected to the tissue interface through the dressing interface.

56. (canceled)

57.-72. (canceled)
